(19) United States
(12) Patent Application Publication
(10) Pub. No.: US 2010/0094418 A1
(43) Pub. Date: Apr. 15, 2010

(54) METHOD FOR PREPARING A COMPOSITE MATERIAL, RESULTING MATERIAL AND USE THEREOF

(75) Inventors: Rachid Zenati, Neuville Sur Saone (FR); Elodie Pacard, Lyon (FR)

Correspondence Address: CANTOR COLBURN, LLP, 20 Church Street, 22nd Floor, Hartford, CT, 06103, US

(73) Assignee: NORAKER, VILLEURBANNE (FR)

(21) Appl. No.: 12/527,080

(22) PCT Filed: Feb. 13, 2008
(86) PCT No.: PCT/FR08/00183

§ 371 Date: Oct. 5, 2009

(30) Foreign Application Priority Data
Feb. 15, 2007 (FR) 0701107

(51) Int. Cl. A61F 2/02; A61K 47/34; B29C 39/36

(52) U.S. Cl. 623/11.11; 523/115; 264/319; 264/328.2

(57) ABSTRACT

The invention relates to a method for preparing a composite material having a homogeneous composition, containing at least one bioactive ceramic phase and at least one bioresorbable polymer. The inventive method is characterised in that it comprises the following steps: a) a bioactive ceramic phase in powder form is obtained, b) the bioactive ceramic powder is suspended in a solvent, c) a bioresorbable polymer is added to the suspension obtained in (b) and mixed to produce a viscous homogeneous dispersion of said bioactive ceramic powder in a solution formed by the solvent and the polymer, and d) the dispersion obtained in (c) is precipitated in an aqueous solution in order to obtain a homogeneous composite material. The invention also relates to the resulting composite material and to the use thereof in the production of implantable medical devices.

### TECHNICAL FIELD OF THE INVENTION

The invention relates to a method for preparing a composite material having a uniform composition, comprising a bioactive ceramic phase and at least one bioresorbable polymer. The invention also relates to an implantable medical device fabricated from this material, in particular by injection molding, injection transfer molding, compression molding, extrusion molding or even by microtechnical machining.

### BRIEF DISCUSSION OF RELATED ART

In the medical field, in particular in the field of implantable medical devices for bone replacement applications, attempts are increasingly being made to obtain implantable medical devices which are bioresorbable in the human or animal body, with advantageous biological and mechanical properties.

In the present application, “bioresorbable” means the property whereby a material is absorbed by the biological tissues and disappears in vivo after a given period, for example in less than 24 months, or even in less than 8 weeks, or even less than a few days.

This is because since these implantable medical devices are required to be in contact with bone, for example in the case of bone replacement, it is advisable that they have biological properties such as osteoconduction or osseo-integration, that is the capacity to promote the growth of the osteoblast cells.

However, considering their function, for example of substitution or fixation, these implantable medical devices must also have very good mechanical strength. Moreover, in the case in which these implantable medical devices are means for fixing other implantable medical devices, such as for example fixation screws, they must not damage the devices which they are required to fix nor the neighboring human tissues, regardless of their shape.

For this purpose, an attempt has been made to prepare composite materials based, on the one hand, on a bioresorbable organic phase and, on the other hand, a ceramic phase.

In the context of the present application, ceramic phase means a mineral phase selected from the group comprising ceramics, vitroceramics, glasses and mixtures thereof.

In such a composite material, the purpose of the ceramic phase is to impart mechanical strength and the necessary biological properties. Thus, it is necessary for this ceramic phase to be present in the composite material in a sufficient minimum content.

Document U.S. Pat. No. 5,977,204 thus describes a composite material comprising an organic phase and a ceramic phase, where the latter may represent from 10 to 70% by volume, based on the volume of the material.

Document U.S. Pat. No. 4,192,021 describes a solid composite material comprising an organic phase and a ceramic phase, in which the quantitative proportion of mineral phase with regard to the organic phase is between 10:1 and 1:1.

However, these prior art materials are generally in the form of nonuniform solid compounds. Such materials are not satisfactory when used for the fabrication of implantable medical devices, in particular of implantable medical devices having complex shapes, such as fixation screws for example. In particular, such materials are very difficult to process by known processing methods, such as injection, injection transfer, compression or extrusion molding. This is because in attempting to process these materials by one of these methods, the organic phase and ceramic phase are generally segregated during the pre-processing heating operation. Thus, the ceramic phase generally separates from the organic phase, thereby preventing normal operation of the machine: this makes it impossible to obtain the desired product.

Thus, the need subsists for a composite material comprising a bioresorbable organic phase and a ceramic phase, said composite material having a uniform composition for the fabrication of implantable medical devices, in particular implantable medical devices of complex shape, for example by processing methods requiring a preheating step, said ceramic phase being present in said composite material in a sufficient quantity to guarantee for the resulting implantable medical devices the biological and mechanical properties required for the function that they are required to perform during their use, for example as fixation and anchoring elements, in the case of interference screws, for implanted bone replacements.

### BRIEF SUMMARY OF THE INVENTION

The present invention remedies this need by proposing a novel composite material and the method for fabricating such a material, this material being suitable for processing, in particular by processing methods requiring a prior step of heating of said material, to produce implantable medical devices, in particular implantable medical devices of complex shape, in such a way that the implantable medical devices thus produced have particularly advantageous biological and mechanical properties for their use in the medical field, for example as strong and resorbable fixation elements for bone replacements.

### DETAILED DESCRIPTION OF THE INVENTION

The present invention relates to a method for preparing a composite material having a uniform composition, comprising at least one bioactive ceramic phase and at least one bioresorbable polymer, characterized in that it comprises the following steps:

a) a bioactive ceramic phase in powder form is obtained,

b) said bioactive ceramic powder is suspended in a solvent,

c) a bioresorbable polymer is added to the suspension obtained in b) and mixed to produce a viscous uniform dispersion of said bioactive ceramic powder in a solution formed by said solvent and said polymer,

d) the dispersion obtained in c) is precipitated in an aqueous solution in order to obtain a uniform composite material.

In the present application, “uniform composition” concerning the composite material or the viscous dispersion means the fact that the various components are uniformly distributed within the volume formed by said composite material or said viscous dispersion. In particular, in the composite material of the invention, the ceramic phase is preferably in the form of particles and the organic phase, that is the polymer, is preferably in the form of a matrix, the ceramic particles being dispersed uniformly within the organic matrix.

In the context of the present application, “bioactive” material means a material capable of developing a biological response at the interface between said material and the human tissues, and therefore of developing a bond between said material and said human tissues.

In the context of the present invention “bioactive glass” means an amorphous glass, partially or totally recrystallized, compatible with the human or animal body, and bioactive in the sense as mentioned above.

The present invention further relates to a composite material having a uniform composition obtainable by the method described above. Preferably, the composite material according to the invention comprises at least 5% by weight, preferably 30% to 80% by weight, of a bioactive ceramic phase, of the total weight of the material.

Preferably, this composite material is in the form of granules.

In the present application, “granule” means a solid particle, porous or not, having a substantially spherical shape. Preferably, the granules according to the invention have an average diameter of 0.1 to 5 mm, preferably between 0.3 and 2 mm.

The composite material according to the invention, in particular when it is in the granule form, is particularly suitable for processing by processing or shaping techniques requiring at least one step of heating of said material, such as injection, injection transfer, compression or extrusion molding.

The present invention further relates to the use of a composite material as described above for fabricating implantable medical devices by a processing technique requiring at least one step of heating of said material.

The present invention further relates to a method for fabricating an implantable medical device, characterized in that it comprises the following steps:

1) a composite material as described above is obtained,

2) said composite material is heated to obtain a uniform paste,

3) said uniform paste is poured into a mold,

4) after cooling, the implantable medical device is obtained by stripping.

The present invention further relates to implantable medical devices obtainable by such a method. These medical devices may comprise at least 30%, preferably between 50 and 80%, by weight of a bioactive ceramic phase, of the total weight of the implantable medical device.

The composite material according to the invention serves to use the techniques of injection molding, injection transfer molding, compression molding, extrusion molding or microtechnical machining, to shape the implantable medical devices, preferably bioresorbable, of all shapes, even the most complex. Due to the particularly uniform composition of the composite material according to the invention, the implantable medical devices obtained have particularly the advantageous biological and mechanical properties for their use in the medical field, in particular in the field of orthopedic surgery of the rachis, the cranial-maxilofacial, dental and traumatology.

The implantable medical devices according to the invention obtained from the composite material according to the invention have in particular a particularly high proportion of ceramic phase. Such implantable medical devices thus have particularly high mechanical strength. It is thus possible to prepare implantable medical devices, preferably bioresorbable, of all shapes, even complex, and to use these implantable medical devices, preferably bioresorbable, for their mechanical properties, for example as fixation and anchoring elements. In particular, it is possible to prepare resorbable implantable medical devices, such as interference screws, pines, cervical and lumbar intervertebral cages, cervical plates, anchors and clips.

The implantable medical devices according to the invention obtained from the composite material according to the invention also have outstanding biological properties: in particular, due to their high ceramic phase content, they are capable of promoting osteoconduction and/or osseo-integration.

According to a first step of the method for preparing the composite material according to the invention, that is step a), a bioactive ceramic phase is obtained in powder form. This bioactive ceramic phase may be selected from ceramics, vitroceramics, bioactive glasses and mixtures thereof. Preferably, the bioactive ceramic phase is a bioactive glass.

In one embodiment of the invention, the bioactive glass consists of 45% by weight of SiO₂, of the total weight of the bioactive glass, 24.5% by weight of CaO, of the total weight of the bioactive glass, 24.5% by weight of Na₂O, of the total weight of the bioactive glass and 6% by weight of P₂O₅, of the total weight of the bioactive glass. This bioactive glass has a property of developing on its surface, when immersed in a physiological medium, a hydroxyapatite carbonate layer (HAC) of the apatite family. Hydroxyapatite carbonate has a structure similar to the mineral portion of the bone. This bioactive glass particularly promotes bone formation. This bioactive glass further comprises components necessary for bone growth, calcium and phosphorus ions in particular.

Such a bioactive glass can be obtained by the conventional method described below: powders of SiO₂, CaCO₃, Na₂CO₃ and P₂O₅ are weighed and mixed. The mixtures are then placed in platinum crucibles and heated to 950° C. in a furnace for the first synthesis step, that is decarbonation, which lasts about 5 hours. This is followed by a second step, that is the melting of the mixtures, which takes place at 1400° C. for a period of about 4 hours. The mixture obtained is then quenched in water. The bioactive glass thus obtained can be ground and screened. Preferably, bioactive glass having an average particle size of 1 to 15 microns, preferably 3 to 4 microns, is used according to the present invention. The density of the bioactive glass is preferably between 2.55 and 2.70 g/cm³, even more preferably between 2.65 and 2.68 g/cm³.

A bioactive glass suitable for the present invention is available on the market under the trade name “45S5®” from USBiomaterials Corporation.

In another embodiment of the invention, the bioactive ceramic phase comprises calcium β-tri-phosphate or hydroxyapatite.

According to the inventive method, the bioactive ceramic phase is prepared in powder form. For this purpose, the raw materials constituting this phase are ground as required, using conventional grinding techniques, to obtain particles. Preferably, the powder of the bioactive ceramic phase has a particle size distribution of 1 to 15 microns, preferably of 3 to 4 microns.

In a second step of the inventive method, that is step b), the bioactive ceramic phase powder is suspended in a solvent. The solvent of step b) may be selected from the group comprising chloroform, acetone, and mixtures thereof. Preferably, the solvent of step b) is acetone.

The suspension of step b) can be prepared conventionally, by simple mixing, for example using a mechanical mixture such as the “IKA® RW 20” type propeller stirrer from IKA-WERKE GMBH & CO.KG, or even with magnetic stirring. Preferably, the suspension step is carried out at ambient temperature (about 20° C.)

In a third step of the inventive method, that is step c), a bioresorbable polymer is added to the suspension obtained in b), and mixed until a uniform viscous dispersion of said ceramic powder is obtained in a solution comprising said solvent and said polymer.

The bioresorbable polymer may be selected from the group comprising polymers of polylactic acid, copolymers of polylactic acid, polymers of polyglycolic acid, copolymers of polyglycolic acid and mixtures thereof.

In an embodiment of the invention, said bioresorbable polymer is a copolymer of poly(L-lactic-co-D,L-lactic) acid. Preferably, said copolymer of poly(L-lactic-co-D,L-lactic) acid comprises 70% of poly(L,lactic) acid and 30% of a 50/50 racemic mixture of poly(D,-lactic) acid: a copolymer having such a composition is available on the market under the trade name “Resomer LR 706®” from Bohringer.

The bioresorbable polymer may be added to said suspension a proportion of 1 to 90% by weight, preferably of 5 to 80% by weight, of the weight of the mixture consisting of the ceramic phase and the bioresorbable polymer.

Thus, preferably, the proportion of ceramic phase in the composite material obtained by the inventive method may be up to 80% by weight of the weight of the composite material.

In an embodiment of the invention, the bioresorbable polymer is added in the form of a powder having a particle size distribution of 800 to 2000 microns.

Preferably, step c) is carried out with stirring, for example with mechanical or magnetic stirring. Preferably, this stirring must allow the total solubilization of the bioresorbable polymer in the solvent. Thus, preferably, the stirring is continued until the total solubilization of the bioresorbable polymer in the solvent: for example, in the case in which the bioresorbable polymer has been added in powder form to the suspension obtained in step b), the stirring is preferably continued until the total solubilization of the bioresorbable polymer particles in the solvent. Preferably, the stirring also allows the homogenization of the complete mixture, that is the solvent, the ceramic powder and the bioresorbable polymer. Thus, preferably, the complete solubilization and homogenization serves to obtain a viscous dispersion, that is of particles of bioactive ceramic phase, for example of bioactive glass, in suspension in a solution comprising solvent and bioresorbable polymer.

In a preferred embodiment of the invention, the stirring is continued until a viscous dispersion is obtained substantially having the consistency of a honey flowing at ambient temperature (about 20° C.)

This homogenization of the dispersion and such as a viscous nature of the dispersion, in particular obtained thanks to the specific order of the steps a)-c), that is a) then b) then c), of the inventive method, serve to ultimately obtain a very uniform composite material, that is in which the ceramic particles are uniformly and regularly distributed in the polymer phase, as clearly appears from the description of FIGS. 2 and 5 below.

In a fourth step of the inventive method, the dispersion obtained in c) is precipitated in an aqueous solution to obtain a uniform composite material. The solvent of steps b) and c) is generally removed during the precipitation step, for example by evaporation.

The composite material obtained by the inventive method preferably comprises at least 5% by weight, preferably 30% to 80% by weight, of a bioactive ceramic phase, of the total weight of the material.

In an embodiment of the inventive method, the dispersion is precipitated in the form of a cluster in water, for example by pouring the dispersion obtained in c) into water tanks. Preferably, the cluster of composite material obtained then being dried and ground to obtain granules. Preferably, the dried cluster is ground to obtain granules having an average diameter of 0.1 to 2 mm, preferably of 0.3 to 1 mm.

In another embodiment of the inventive method, said dispersion is precipitated in the form of droplets in water, said droplets of composite material obtained then being dried to obtain granules. The granules directly obtained by precipitation of droplets preferably have a substantially spherical shape. Alternately, the dried droplets may be ground to obtain granules.

The granules thus obtained preferably have an average diameter of 0.1 to 5 mm, preferably of 1 to 2 mm.

Step d) of precipitation by the wet method may thus comprise a step of pouring the dispersion obtained in c) into a burette, provided with a cock and the installation of a drip system above a water tank.

Thus, the granules obtained by the inventive method, by precipitation of a cluster or droplets, preferably comprise at least 5% by weight, preferably 30% to 80% by weight, of a ceramic phase, of the total weight of the granule.

The composite material and/or the granules obtained by the inventive method have a uniform composition, that is, the particles of ceramic phase, for example of bioactive glass, are dispersed very uniformly in the bioresorbable polymer matrix.

FIGS. 2 and 5, (see also examples 1, 2 and 3) are scanning electron microscope pictures of the composite material according to the invention with various ceramic phase and polymer phase compositions, showing the distribution of the bioactive ceramic particles in the polymer matrix. It appears that the bioactive ceramic particles are distributed uniformly throughout the matrix. In particular, thanks to the inventive method, the precipitation in step d) of the uniform dispersion obtained in c) in water enables the ceramic phase particles to be chemically bound to the bioresorbable polymer matrix and not only mechanically, as in the prior art materials.

It is thus possible to fabricate powdery compositions of granules of uniform composite material obtained by the inventive method and to use the composite material of the invention either directly, or in the form of such powdery compositions, in processing techniques requiring a preheating step, without causing the separation or segregation in the composite material or the granules of composite material between the ceramic phase and the organic phase of bioresorbable polymer during this preheating step.

The composite material according to the invention, in particular in the form of granules, may be used effectively in shaping methods by techniques requiring at least one step of heating of said material to fabricate implantable medical devices.

Thus, the implantable medical devices according to the invention may be fabricated by the following fabrication method:

1) a composite material is obtained as described above,

2) said composite material is heated to obtain a uniform paste,

3) said uniform paste is poured into a mold,

4) after cooling, the implantable medical device is obtained by stripping.

During step 2) of heating the composite material according to the invention, the temperature, the temperature may rise from 130 to 170° C. Due to the particularly uniform nature of the composition of the composite material according to the invention, the composite material, under the action of heat, is converted to a paste which itself remains uniform. No separation or segregation occurs of the phases, ceramic on the one hand, and polymer on the other hand.

For example, step 3) may be carried out by a processing technique selected from injection molding, injection transfer molding, compression molding, extrusion molding. During this step, the paste obtained in step 2), since it is particularly uniform, is perfectly suitable for being treated by the machine of the technique considered, for example the die of the extruder.

An implantable medical device is thereby obtained having outstanding biological and mechanical properties. This implantable medical device may for example be in the form of an interference screw, a pine, cervical and lumbar intervertebral cages, cervical plates, anchors or clips.

The method for preparing the composite material according to the invention and the composite material according to the invention serve to fabricate implantable medical devices that are particularly strong, bioresorbable and promote bone formation.

The composite material according to the invention, in particular in the form of granules, may be shaped by injection molding, injection transfer molding, compression molding, extrusion or microtechnical machining, to fabricate bioresorbable implantable medical devices having complex shapes, for implantation in the human or animal body, such as for example interference screws, pines, cervical and lumbar intervebtebtral cages, cervical plates, anchors, clips, etc.

### BRIEF DESCRIPTION OF THE DRAWINGS

Examples illustrating the present invention will now be provided, in which:

FIG. 1 is a micrograph of a granular composite material obtained by the inventive method, comprising 50% by weight of ceramic phase and 50% by weight of polymer phase, of the weight of the granule, obtained by scanning electron microscope (840 A LGS from JEOL) with a magnification of 25.

FIG. 2 is a micrograph of the surface of the granule of FIG. 1 obtained by scanning electron microscope (840 A LGS from JEOL) with a magnification of 350.

FIG. 3 is a view obtained by scanning electron microscope (840 A LGS from JEOL) with a magnification of 650, of the granule of FIG. 1 covered with cells (MG-63 osteoblasts).

FIG. 4 is a micrograph of a granule of composite material obtained by the inventive method, comprising 75% by weight of ceramic phase and 25% by weight of polymer phase, of the weight of the granule, obtained by scanning electron microscope (840 A LGS from JEOL) with a magnification of 26.

FIG. 5 is a micrograph of the surface of the granule of FIG. 4 obtained by scanning electron microscope (840 A LGS from JEOL) with a magnification of 147.

FIG. 6 is a view obtained by scanning electron microscope (840 A LGS from JEOL) with a magnification of 800, of the granule of FIG. 4 covered with cells (MG-63 osteoblasts).

### EXAMPLES OF THE INVENTION

### Example 1

### Preparation of a Composite Material Having a Uniform Composition According to the Invention Comprising 50% by Weight of Ceramic Phase and 50% by Weight of Polymer Phase of the Weight of the Composite Material

The bioactive ceramic phase consists of a powder of bioactive glass comprising 45% of SiO₂, 24.5% of CaO and Na₂O and 6% of P₂O₅ in mass percentage.

This bioactive glass is obtained by the following preparation method: powders of SiO₂, CaCO₃, Na₂CO₃ and P₂O₅ are weighed and mixed. The mixtures are then placed in platinum crucibles and heated to 950° C. in a furnace for the first synthesis step, that is decarbonation, which lasts about 5 hours. This is followed by a second step, that is the melting of the mixtures, which takes place at 1400° C. for a period of about 4 hours. The mixture obtained is then quenched in water.

The bioactive glass thus obtained can be ground and screened having an average particle size of 3 to 4 microns. The density of the bioactive glass is preferably between 2.65 and 2.68 g/cm³.

100 g of the bioactive glass particles thus obtained are placed in suspension in acetone.

A copolymer is obtained of copolymer of poly(L-lactic-co-D,L-lactic) acid. Preferably, said copolymer of poly(L-lactic-co-D,L-lactic) acid comprises 70% of poly(L,lactic) acid and 30% of a 50/50 racemic mixture of poly(D,-lactic) acid: a copolymer having a such composition is available on the market under the trade name “Resomer LR 706®” from Bohringer. 100 g of this copolymer poly(L-lactic-co-D,L-lactic) acid are placed in solution in the suspension of bioactive glass and mixed using a stirrer or a mixer for 5 hours until complete solubilization of the copolymer in the acetone. After 5 h, a viscous dispersion having the consistency of a honey flowing at ambient temperature (about 20° C.) and very uniform, is obtained.

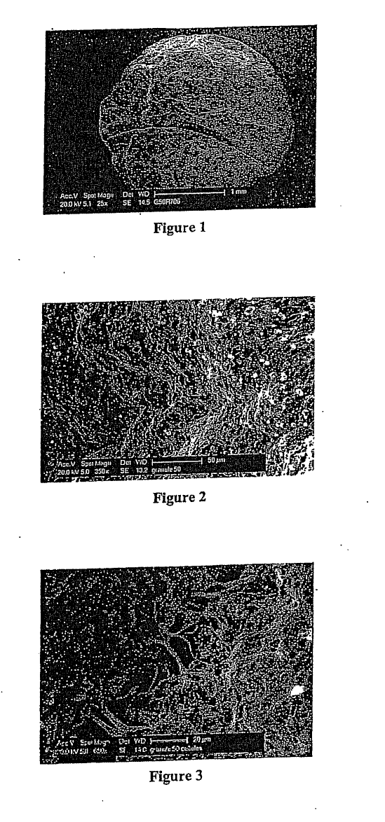

The viscous dispersion is then poured into a burette provided with a cock. The flow rate of the dispersion is adjusted by the cock in order to obtain a drip. The droplets are precipitated in water, a step during which the acetone is removed by evaporation. The granules obtained by the precipitation of the droplets are then dried in ambient atmosphere (about 20° C.) for 2 h and then in an oven at 40° C. for 24 h. Granules having a substantially spherical shape are obtained: such a granule can be seen in FIG. 1, which is a micrograph of such a granule obtained with a type 840 A LGS scanning electron microscope from JEOL with magnification of 25.

These granules of composite material have a uniform composition. The uniform composition of these granules is shown in FIG. 2, which is a micrograph of the surface of such a granule obtained with a type 840 A LGS scanning electron microscope from JEOL with a magnification of 350. In this micrograph, the bioactive glass particles clearly appear in the form of small white spots, uniformly dispersed in the polymer matrix. Each granule has a composition of 50% by weight of bioactive glass in the form of particles and 50% by weight of bioresorbable polymer in the form of a matrix in which said bioactive glass particles are uniformly distributed.

These granules have a particle size distribution, or an average granule size, of 1 to 2 mm.

It was confirmed by absolute density measurements by helium pycnometer (Micromeritics Accu Pyc 1330) that the granules thus obtained are uniform and shown in Table 1 below.

TABLE 1
Absolute density and calculated density as a
function of the mass percentage of bioactive glass in
the granule.

| | Mass percentage “Resomer LR 706 ®”/Bioactive glass | | | | | |
|---|---|---|---|---|---|---|
| | 100/0 | 80/20 | 50/50 | 40/60 | 25/75 | 0/100 |
| Absolute | 1.27 | 1.47 | 1.76 | 1.89 | 2.17 | 2.67 |
| density (g/cm³ | | | | | | |
| measured) | | | | | | |
| Calculated | 1.27 | 1.42 | 1.72 | 1.85 | 2.09 | 2.67 |
| density | | | | | | |
| (theoretical | | | | | | |
| g/cm³) | | | | | | |

In the composite material obtained, the composition of bioactive glass measured, that is the proportion of bioactive glass in the final composite material, varies only slightly, that is, closely similar to the composition of bioactive glass expected from the density calculated from the starting proportions of bioactive glass and bioresorbable polymer. A variation of 0 to 3% was observed in the case of mixtures of the polymer “Resomer LR 706®/bioactive glass of 80/20; 50/50; 40/60; 25/75 in mass percentage.

These results were confirmed by calculation from the mixing law:

$$\frac{1}{{ρ}_{eq}} = \frac{{x}_{b}}{{ρ}_{b}} + \frac{{x}_{p}}{{ρ}_{p}}$$

where:

ρ_{eq}: absolute density of the mixture (g/cm³)

ρ_{b}: density of bioactive glass=2.67 g/cm³

ρₚ: density of polymer “Resomer LR 706®”=1.27 g/cm³

X_{b} : mass percentage of bioactive glass (%)

Xₚ: mass percentage of polymer “Resomer LR 706®” (%)

It was confirmed that the granules thus obtained have a bioactive character. Thus, these granules were immersed in a SBF solution (Simulated Body Fluid) containing the same ions in the same concentrations as human plasma, having a pH of 7.2 to 7.4.

X-ray diffraction and scanning electron microscope analyses show, after immersion of the granules in SBF, the formation of a hydroxyapatite phase crystallized on the surface of the granules. The formation of this phase is characteristic of the bioactivity of the granules.

Furthermore, scanning electron microscope observations serve to confirm that the cells (MG-63 osteoblasts) adhere to the surface of the granules: the adhesion of the cells can be seen in FIG. 3, which is a view obtained with a type 840 A LGS scanning electron microscope from JEOL, with a magnification of 650, of a granule of the present example 1 covered with cells. These cells form cytoplasmic extensions at the level differences of the granules.

Thus, the granules obtained in this example 1 are particularly suitable for fabricating implantable medical devices having complex shapes, by processing techniques requiring a prior heating step. The implantable medical devices obtained with the granules of example 1 comprise 50% by weight of ceramic phase. They accordingly have outstanding biological mechanical properties which are particularly advantageous for use as fixation elements of bone replacements, for example.

### Example 2

### Preparation of a Composite Material Having a Uniform Composition According to the Invention Comprising 75% by Weight of Ceramic Phase and 25% by Weight of Polymer Phase of the Weight of the Composite Material

A viscous dispersion was prepared by the method of example 1, using the same bioactive glass as in example 1 and the same bioresorbable polymer as in example 1, but by respectively using 75 g of bioactive glass and 25 g of bioresorbable polymer.

The viscous dispersion was then poured directly into a water tank to obtain a cluster of precipitate comprising bioactive glass and bioresorbable polymer. The cluster of composite material obtained after this precipitation has a uniform composition, visible in FIG. 5, which is a micrograph of the surface of such a granule obtained with a type 840 A LGS scanning electron microscope from JEOL with a magnification of 147. In this micrograph, the bioactive glass particles clearly appear in the form of small white spots, uniformly dispersed in the polymer matrix. This cluster has a composition of 75% by weight of bioactive glass in the form of particles and 25% by weight of bioresorbable polymer in the form of a matrix in which said bioactive glass particles are uniformly distributed.

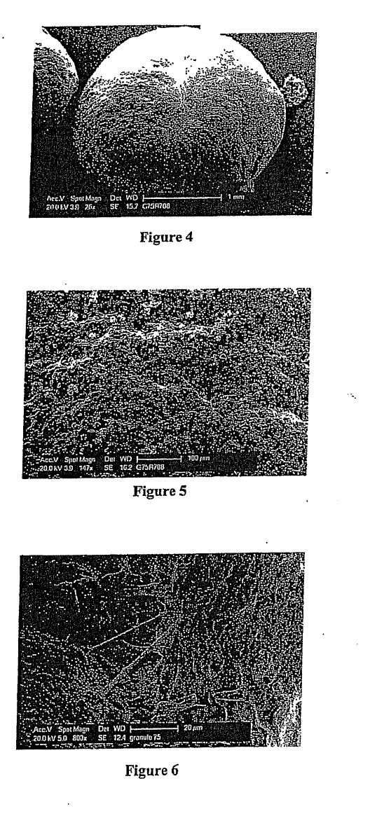

This cluster was then dried and ground. Granules of composite material were thus obtained comprising bioactive glass and bioresorbable polymer, having a particle size distribution, that is an average granule size, of 300 to 2000 microns. Granules having a substantial spherical shape are thereby obtained. Such a granule is shown in FIG. 4, which is a micrograph of such a granule obtained with a type 840 A LGS scanning electron microscope from JEOL with a magnification of 26.

The bioactivity of these granules was checked and confirmed in the same way as in example 1. In particular, scanning electron microscope observations serve to confirm that the cells adhere to the surface of the granules: the adhesion of the cells can be seen in FIG. 6, which is a view obtained with a type 840 A LGS scanning electron microscope from JEOL with a magnification of 800, a granule of the present example 2 covered with cells. These cells form cytoplasmic extensions at the level differences of the granules.

### Example 3

### Fabrication of an Implantable Medical Device from the Granules Obtained in Example 1

A powder compositions of granules of composite material obtained in example 1 was poured into a transfer bowl. The composition there was mixed and heated. Thanks to the particularly uniform nature of the composition of the granules of example 1, the mechanical and heat treatment supplied a soft paste which remained uniform. This uniform, bubble-free paste was transferred in a mold toward an orifice. The paste was thrust by pressure through an orifice by a piston and filled a closed and cooled mold. In contact with the cold walls, the paste assumed the shape of the mold and solidified. The mold was then opened to extract the piece.

After stripping, finished or semi-finished products having complex shapes are obtained in a single operation.

In the same way, a powdery composition of granules of composite material having a uniform composition according to the invention and comprising 20% by weight of ceramic phase and 80% by weight of polymer phase of the weight of the composite material was used to fabricate an implantable medical device by injection transfer molding or injection molding.

For example, cervical plates, cervical and lumbar intervertebral cages, fixation screws and interference screws are fabricated by injection transfer molding or injection molding with the following operating conditions:

### Processing Parameters:

### Pressure: 90-110 bar

### Temperature: 135-165° C.

### Mechanical Properties of the Products Obtained:

Compression tests were performed on an Instron machine on specimens having the dimensions 10 mm×10 mm×4 mm (according to standard ISO 604) of composite materials obtained according to example 1 with a crossbeam speed of 0.5 mm/min. The results obtained are given in Table 2 below:

TABLE 2
Compression properties for composite
materials according to the invention having a
decomposition of 20% by weight of bioactive glass/80%
of “Resomer LR706 ®”, 50% by weight of bioactive
glass/50% by weight of “Resomer LR706 ®”, and the
polymer “Resomer LR706 ®” alone.

| | Material | | | |
|---|---|---|---|---|
| | Tested | | | |
| | | 20/80 | 50/50 | |
| | | (Bioactive | (Bioactive | |
| | “Resomer | glass/“Resomer | glass/“Resomer | Cortical |
| | LR706 ®” | LR706 ®” | LR706 ®” | bone |
| Young's | 4 | 6 | 10 | 20* |
| modulus | | | | |
| (GPa) | | | | |
| Yield | 80 | 87 | 84 | — |
| stress | | | | |
| (MPa) | | | | |
| Compressive | 84 | 140 | 149 | 150* |
| breaking | | | | |
| stress | | | | |
| (MPa) | | | | |

*Reference value reported in the literature

The measurements of Young's modulus were taken by the resonance method using a Grindo Sonic type of instrument. This method uses the principle of excitation by impact. The energy acquired by a part loaded by an impact is dissipated in the form of vibrations which depend, among other factors, on the properties of the material. The measurement of the natural resonance frequency of test specimens having a simple geometry serves to determine the modulus.

The values obtained for Young's modulus and the compression tests show that the composite material according to the invention has better mechanical properties than the polymer alone. The higher the bioglass concentration in the composite, the higher the Young's modulus. The composite material according to the invention has mechanical properties close to those of the bone, both in elasticity (Young's modulus) and in compressive strength. The composite material according to the invention has a mechanical strength of about 149 MPa, very similar to that of the cortical bone (150 MPa).

1. A method for preparing a composite material having a uniform composition, comprising at least one bioactive ceramic phase and at least one bioresorbable polymer, the method comprising:
a) obtaining a bioactive ceramic phase in powder form,
b) suspending said bioactive ceramic powder in a solvent,
c) adding a bioresorbable polymer to the suspension obtained in b) and mixing to produce a viscous uniform dispersion of said bioactive ceramic powder in a solution formed by said solvent and said polymer, and
d) precipitating the dispersion obtained in c) in an aqueous solution in order to obtain a uniform composite material.

2. The method as claimed in claim 1, wherein, during step d), said dispersion is precipitated in the form of a cluster in water, the cluster of composite material obtained then being dried and ground to obtain granules.

3. The method as claimed in claim 1, wherein, during step d), said dispersion is precipitated in the form of droplets in water, said droplets of composite material obtained then being dried to obtain granules.

4. The method as claimed in claim 3, wherein the dried droplets are ground to obtain granules.

5. The method as claimed in claim 1, wherein said bioactive ceramic phase is selected from ceramics, vitroceramics, bioactive glasses and mixtures thereof.

6. The method as claimed in claim 1, wherein said bioactive ceramic phase is a bioactive glass.

7. The method as claimed in claim 1, wherein said bioactive glass comprises 45% by weight of SiO₂, of the total weight of the bioactive glass, 24.5% by weight of CaO, of the total weight of the bioactive glass, 24.5% by weight of Na₂O, of the total weight of the bioactive glass and 6% by weight of P₂O₅, of the total weight of the bioactive glass.

8. The method as claimed in claim 1, wherein the powder of the bioactive ceramic phase of step a) has a particle size distribution from 1 to 15, preferably from 3 to 4 microns.

9. The method as claimed in claim 1, wherein the solvent of step b) comprises at least one of chloroform, acetone and mixtures thereof.

10. The method as claimed in claim 1, wherein said bioresorbable polymer comprises at least one of polymers of polylactic acid, copolymers of polylactic acid, polymers of polyglycolic acid, copolymers of polyglycolic acid and mixtures thereof

11. The method as claimed in claim 1, wherein said bioresorbable polymer is a copolymer of poly(L-lactic-co-D,L-lactic) acid.

12. The method as claimed in the preceding claim 1, wherein said polymer of poly(L-lactic-co-D,L-lactic) acid comprises 70% of poly(L,lactic) acid and 30% of a 50/50 racemic mixture of poly(D,-lactic) acid.

13. The method as claimed in claims 1, wherein said bioresorbable polymer is added in step c) in a proportion of 1 to 90% by weight, of the weight of the mixture consisting of the ceramic phase and the bioresorbable polymer.

14. The method as claimed claims 1, wherein step c) is carried out with stirring.

15. The method as claimed in claim 1, wherein the stirring is continued until the total solubilization of the bioresorbable polymer in the solvent.

16. The method as claimed in claim 14, wherein the stirring is continued until a viscous dispersion is obtained substantially having the consistency of a honey flowing at ambient temperature.

17. The method as claimed in claim 3, wherein step d) comprises a step of pouring of the dispersion obtained in c) into a burette provided with a cock and installation of a drip system above a water tank.

18. A composite material having a uniform composition obtainable by The method as claimed in claim 1.

19. The composite material as claimed in claim 18, wherein it comprises at least 5% by weight of a bioactive ceramic phase, of the total weight of the material.

20. The composite material as claimed claim 18, wherein it is in the form of granules.

21. The composite material as claimed in claim 20, wherein the granules have a particle size distribution of 0.1 to 5 mm.

22. A method for fabricating implantable medical devices by a processing technique requiring at least one step of heating of said composite material of claim 15.

23. A method for fabricating an implantable medical device, comprising:
1) obtaining a composite material as claimed in claim 18,
2) heating said composite material is-heated-to obtain a uniform paste,
3) pouring said uniform paste into a mold,
4) obtaining, after cooling, the implantable medical device by stripping.

24. The method as claimed in claim 23, wherein step 3) is carried out by a processing technique selected from injection molding, injection transfer molding, compression molding, extrusion molding.

25. An implantable medical device obtainable by a method as claimed in claim 23.

26. The implantable medical device as claimed in claim 25, wherein it comprises at least 30% by weight of a bioactive ceramic phase, of the total weight of the implantable medical device.

27. The implantable medical device as claimed in claim 25, wherein it is the form of an interference screw, a pine, cervical and lumbar intervertebral cages, cervical plates, anchors or clips.
